(19) United States
(12) Patent Application Publication
(10) Pub. No.: US 2011/0184441 A1
(43) Pub. Date: Jul. 28, 2011

(54) Prosthetic repair patch with integrated sutures and method therefor

(76) Inventor: Pascal St-Germain, Quebec (CA)

(21) Appl. No.: 13/064,575

(22) Filed: Apr. 1, 2011

Related U.S. Application Data
(63) Continuation-in-part of application No. 11/651,504, filed on Jan. 10, 2007.

(51) Int. Cl. A61B 17/00; A61B 17/04

(52) U.S. Cl. 606/151; 606/228

(57) ABSTRACT

A prosthetic repair patch has a sheet and a plurality of sutures integrated therewith and laid securely there across. The sheet, with first and second sheet surfaces, completely under covers a hernia in the abdominal tissue of a patient with the first sheet surface adjacently abutting a first surface of the tissue that faces away from a person installing the patch. The sutures are preconnected, prior to packaging and sterilization of the patch, to the sheet in a spaced apart configuration from one another and each has a longitudinal end thereof that extends from the first sheet surface. Each suture end is adapted to extend through the tissue for locally abutting the first sheet surface to the first tissue surface and to extend from an opposite second surface of the tissue for attachment with another suture end thereat for local fastening of the sheet to the tissue. The present invention also discloses a method of under covering a hernia with the repair patch.

### CROSS-REFERENCE TO RELATED APPLICATIONS

The present application is a continuation-in-part (C.I.P.) of application Ser. No. 11/651,504, filed on Jan. 10, 2007, now abandoned.

### FIELD OF THE INVENTION

The present invention relates to prosthetic repair patches for repairing undesired apertures, such as hernias, in biological tissue of the abdominal wall of a patient, and is more particularly concerned with a prosthetic repair patch having integrated sutures and a method therefor.

### BACKGROUND OF THE INVENTION

It is well known in the art to use prosthetic repair patches to repair, by under covering, undesired apertures, such as hernias, in biological tissue of the abdominal wall, aponeurosis or the like of a patient with prosthetic repair patches. Typically, such patches are made of biologically compatible material and are surgically placed under the hernia and then connected to the abdominal wall surrounding the hernia using sutures.

An example of such a prosthetic repair patch is described in U.S. Pat. No. 6,120,539, issued to Eldridge et al. The patch described therein comprises a sheet used for, among other things, repair of ventral hernias, in patients by placement of the patch under the hernia with a first sheet surface thereof in adjacent abutment to the surrounding tissue, typically a first tissue surface which faces away from the health professional that is placing the patch in the patient to repair the hernia. The advantages of using such patches, as opposed to other approaches for repairing hernias, are generally well known in medical arts, and include, notably, reduced risk of hernia reoccurrence. Such patches are typically connected to the surrounding tissue, the abdominal wall in the case of ventral hernias, with sutures. Each suture is generally a biologically compatible thread or fiber having generally opposed first and second ends. The suture is typically inserted by the health professional into the surrounding tissue from a second tissue surface, facing towards the health professional and generally opposite the first tissue surface, through the tissue and the first tissue surface and then through the patch. The suture is then drawn across a portion of a second sheet surface, generally opposite the first sheet surface, and then back through the sheet, the tissue, and the second tissue surface. Thus, there is an intermediate portion, intermediate the ends, extending across a portion of the second sheet surface. The suture, and more specifically the ends thereof, may then be pulled towards the health professional to ensure that the first sheet surface is held locally adjacently abutting the first tissue surface with the ends fastened together. This operation is generally repeated for each suture until the sheet is connected around the entirety of its perimeter to the surrounding tissue with the first sheet surface adjacently abutting the first tissue surface and a portion of the sheet completely covering the hernia. This technique is typically referred to as an underlay repair for a hernia, the advantages of which are well known to one skilled in the medical arts.

Unfortunately, as described above, the use of conventional patches for the underlay hernia repair technique described above obliges the health professional to insert the sutures through the tissue and the sheet of the patch, often with a needle, and then to loop the suture back through the sheet and tissue. As the sheet is placed on the first tissue surface facing away from the health professional, when the suture and needle are inserted through the sheet and tissue, they are often inserted towards subjacent internal organs, which creates a danger that the needle will pierce, and potentially damage, the subjacent internal organs. This may lead to surgical and post-surgical complications, such as, among others, tearing, bleeding (internal hemorrhage) of the internal organs such as intestine or the like and infection thereof (peritonitis, abscess). For example, in the case underlay repair of ventral hernias, the suture and needle are inserted towards the intestine, which poses a risk of damage thereto. Additionally, as the safe passage of the suture through the surrounding tissue and sheet requires careful manipulation of the needle to avoid other portions of non-damaged tissue, the use of conventional patches for the underlay procedure is also time consuming and complex.

Furthermore, a surgeon using the patch described in U.S. Pat. No. 6,383,201, issued to Dong, spends a significant amount of time in connecting the different sutures to the patch just prior insertion of the patch into the patient's body while being next to, or in front of, the patient's body opened at the incisional area ready to receive the patch, essentially for sterilization concerns. All this time significantly increases the surgery time and risks of contamination of the patient. Furthermore, this handling of the patch by the surgeon for preconnection of the sutures increases the risks of contaminating the patch and the sutures which are originally sterilized. Also, preconnecting sutures to the patch in front of the patient would imply that the surgeon has to deal with a plurality of suture ends, each of a length typically varying between about 6 to 8 inches, hanging therefrom while inserting the preconnected patch into the incisional area, thus rendering the operation tremendously complicated and risky, not even considering the fact that further the odds of mixing of the sutures is high, and obviously not recommended.

Insertion of the patch under the damaged region of the damaged tissue often requires access opening(s), or incision(s), through the skin and other surrounding body parts of the patient that may be non-negligible in size and therefore increase the risk of any problem arising to affect the health of the patient.

Conventional installation of patches often leads to non-uniform and unequal attachment of the patch to the abdominal wall all around the hernia, which subsequently leads recurrent patch repair on a same patient.

Accordingly, there is a need for an improved prosthetic replacement patch and method of use thereof that obviate the aforementioned difficulties.

### SUMMARY OF THE INVENTION

It is therefore a general object of the present invention to provide an improved prosthetic replacement patch for repairing hernias in biological tissue of the abdominal wall or the like of a patient and a method therefor.

An advantage of the present invention is that repair of the hernia is simplified and accelerated by using the patch provided by the present invention.

Another advantage of the present invention is that the risk of piercing or damaging other tissue and subjacent internal organs during connection of the patch provided by the present invention to the tissue surrounding the hernia is reduced.

A further advantage of the method using the patch provided by the present invention is that the risk of infection, either to the tissue surrounding the hernia or to other subjacent internal tissue, is reduced by use thereof to repair the hernia.

Still another advantage of the present invention is that the uniform and equal installation and attachment of the patch to the abdominal wall is increased while the risk of recurrence of the hernia is reduced.

Another advantage of the present invention is that the method thereby allows for better placement of the patch compared to any conventional placement method of the patch.

Still another advantage of the method of the present invention is that the surgery time is reduced by eliminating the need to connect sutures to the prosthetic repair patch during surgical procedures, along with the risk of contamination of the patient associated with the surgery time.

Yet another advantage of the method of the present invention is that the surgery time and risks are reduced by having the integrated suture ends at least partially releasably secured to the sheet.

According to a first aspect of the present invention, there is provided a prosthetic repair patch comprising:
a sheet comprising biologically compatible material, the sheet having first and second sheet surfaces and being sized and shaped for completely covering an aperture in biological tissue in a body of a patient with the first sheet surface adjacently abutting a first tissue surface of the tissue, the first tissue surface generally facing away from a person installing the patch; and
a plurality of sutures preconnected and integral to the sheet and at least partially releasably secured thereto, thereby eliminating a need to connect the sutures thereto during surgical procedures, the sutures being preconnected to the sheet in a spaced apart configuration from one another and extending from the first sheet surface, each the suture being adapted to extend through the tissue for locally and adjacently abutting the first sheet surface to the first tissue surface to extend from an opposite second surface of the tissue for attachment with another the suture adjacent the second tissue surface to locally fasten the sheet to the tissue.

In a second aspect of the present invention, there is provided a method for covering an aperture in an internal biological tissue extending therearound in a body of a patient with a prosthetic repair patch comprising a sheet of biologically compatible material and sutures preconnected and integral thereto, and having at least a respective suture longitudinal end extending from a first sheet surface of said sheet and releasably securely laid thereacross, the method comprising the steps of:
a) obtaining the prosthetic repair patch having the sheet of biologically compatible material and sutures preconnected thereto and integral therewith;
b) positioning said sheet proximal a first tissue surface of the tissue in the body with said first sheet surface facing the first tissue surface and said sheet extending under the aperture, the first tissue surface generally facing away from a person installing said patch; and
c) securing said sheet to the tissue.

In one embodiment, the step of obtaining the patch includes taking the patch having the sheet and sutures preconnected thereto and integral therewith out from a sterilized manufacturing package.

Conveniently, the sterilized patch is in a rolled configuration inside the package.

Typically, the step of positioning the sheet includes unrolling the sheet from a compact rolled first sheet configuration into an unrolled second configuration with the first sheet surface facing the first tissue surface.

Typically, the patch includes a visual identifier connected thereto, and wherein the step of positioning the sheet includes visually identifying the visual identifier to orient the sheet relative to the tissue and to the aperture thereunder.

In one embodiment, the sutures are at least partially folded, and rolled or twisted in corresponding pairs across the first sheet surface.

Conveniently, the sutures are at least partially folded, and rolled or twisted in corresponding pairs across the first sheet surface.

Typically, the sutures are at least partially releasably bonded onto and across the first sheet surface.

Conveniently, the patch having the sheet and sutures preconnected thereto and integral therewith are sterilized prior to packaging thereof.

Conveniently, the sutures are at least partially folded, rolled, or twisted in corresponding pairs across the first sheet surface, and are preferably at least partially releasably bonded onto and across the first sheet surface.

In one embodiment, the step of securing the sheet to the tissue includes the steps of:
c) extending each said suture end through the tissue and out from a second tissue surface of the tissue generally opposite the first tissue surface;
d) pulling each said suture end until the first sheet surface locally and adjacently abuts the first tissue surface while under covering the aperture; and
e) attaching each said suture end with another the suture end adjacent the second tissue surface to locally fasten the sheet to the tissue.

Conveniently, the step of extending each suture end includes extending the suture end from a first suture position in which the suture end is securely laid across the first tissue surface into a second suture position in which the suture end is extended for connecting to the tissue.

Typically, the suture ends are arranged in pairs and twisted to one another adjacent the first sheet surface when in the first suture position, the step of extending each the suture end further including, for each the suture pair, the step of:
untwisting the suture pair while extending corresponding the suture ends from the first suture position into the second suture position.

Alternatively, the suture ends are arranged in pairs and rolled adjacent the first sheet surface when in the first suture position, the step of extending each the suture end further including, for each the suture pair, the step of:
unrolling the suture pair while extending corresponding the suture ends from the first suture position into the second suture position.

In one embodiment, the step of extending each the suture end comprises, for each the suture end, the steps of:
c1) inserting a suture passer through the tissue from the second tissue surface through the first tissue surface for engaging the suture end therewith; and
c2) drawing the suture end through the tissue with the suture passer from the first tissue surface toward and out of the second tissue surface.

Other objects and advantages of the present invention will become apparent from a careful reading of the detailed description provided herein, with appropriate reference to the accompanying drawings.

### BRIEF DESCRIPTION OF THE DRAWINGS

Further aspects and advantages of the present invention will become better understood with reference to the description in association with the following Figures, in which similar references used in different Figures denote similar components, wherein:

FIG. 1 is a top perspective view of a prosthetic repair patch in accordance with an embodiment of the present invention, with integrated sutures;

FIG. 2 is top perspective view of biological abdominal tissue having a hernia (aperture) therein and surrounded thereby, with the patch shown in FIG. 1 under covering, and thereby repairing, the aperture;

FIG. 3 is a side sectional view of the abdominal tissue and patch shown in FIG. 2, taken along line 3-3 of FIG. 2;

FIG. 4a is a perspective view of the patch shown in FIG. 1 with the sutures in a first suture configuration laid on a first sheet surface of the patch;

FIG. 4b is a view similar to FIG. 4a showing another embodiment of the present invention with the sutures arranged in groups; and

FIG. 5 is a perspective view of the patch shown in FIG. 4d in a preferably packaged rolled up configuration.

### DETAILED DESCRIPTION OF THE PREFERRED EMBODIMENTS

With reference to the annexed drawings the preferred embodiments of the present invention will be herein described for indicative purpose and by no means as of limitation.

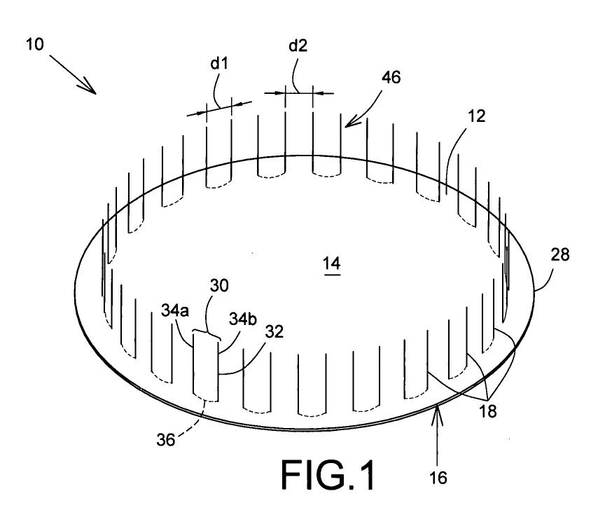

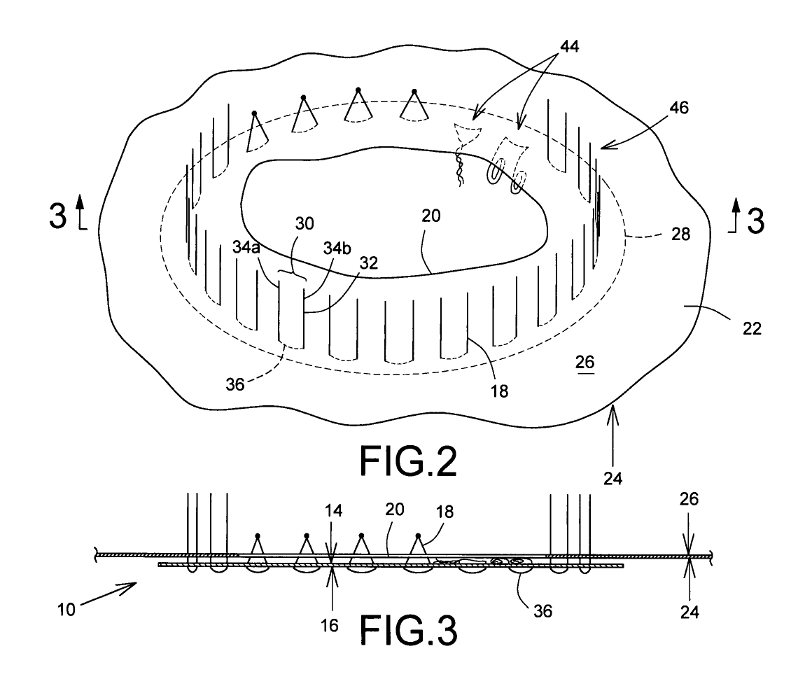

Reference is now made to FIGS. 1 and 2, which show a prosthetic replacement patch, shown generally as 10, in accordance with an embodiment of the present invention for repairing an aperture 20 or hernia in surrounding biological tissue 22 of the abdominal wall of a patient. For the purposes of this description, it should be noted that the term aperture 20 denotes any undesired aperture 20 in biological tissue 22 of a patient, including hernias, tears, punctures, and the like. However, the patch 10 described herein is ideally suited for repair of hernias, and ventral hernias in a particular, using an underlay repair surgical technique. It should also be noted that the term repair, with regard to apertures 20 in the tissue 22, generally denotes, for the purposes of this description, the complete under covering of an aperture 20 with the patch 10 and the connecting of the patch 10 to surrounding tissue 22 surrounding the aperture 20, such that the aperture 20 is completely covered, i.e. closed. However, the patch 10 described herein is particularly suited for use in underlay hernia repair procedures, in which the patch 10 is placed underneath the surrounding abdominal tissue 22 surrounding the aperture 20, i.e. facing a first tissue surface 24 facing away from the health professional placing the patch in the patient, with the patch completely under covering the aperture 20 and sutured to the surrounding tissue 22 on a second tissue surface 26, generally opposite the first tissue surface 24.

The patch 10 has a sheet 12, possibly having multiple layers, and which has a first sheet surface 14 and a second sheet surface 16 comprised of biologically compatible material, suitable for placement within a patient. Such biologically compatible materials typically consist of, for example, polyester, polyglycolic acid, polypropylene, polytetrafluoroethylene, and a combination of polytetrafluoroethylene and polypropylene. However, any biologically compatible material typically suitable for long term or permanent placement within a patient, or eventually resorptive (absorbable), and which is suitable for under covering the aperture 20 in the surrounding biological abdominal tissue 22 may be deployed. The sheet 12 is sized and shaped for completely covering the aperture 20 in the surrounding biological tissue 22 with the first sheet surface 14 adjacently and locally abutting the first tissue surface 24 for closing off, i.e. covering, and repairing the aperture 20.

Referring now to FIGS. 1, 2, and 3, the patch 10 also has a plurality of sutures 18, connected to the sheet 12 in a spaced apart configuration from each other, preferably around the entire perimeter 28 of the sheet 12 and which have at least one, preferably respective both longitudinal end 34a, 34b extending from the first sheet surface 14. The sutures 18, integral to the patch 10, are used to connect the sheet 12 to the tissue 22 to at least partially secure the sheet 12 thereto with the first sheet surface 14 adjacently abutting the first tissue surface 24 for under covering the aperture 20. More specifically, each end 34a, 34b of the sutures 18 are adapted for extension through the tissue 22, from the first tissue surface 24 to the second tissue surface 26, for locally and adjacently abutting the first sheet surface 14 to the first tissue surface 24 with the sutures ends 34a, 34b extending outwardly from the second tissue surface 26 for attachment of each suture end 34a to another suture end 34b adjacent the second tissue surface 26, typically of the same suture 18. Accordingly, the sutures locally fasten the sheet 12 to the tissue 22 with the first sheet surface 14 adjacently abutting the first tissue surface 24 for completely under covering, and thereby repairing, the aperture 20. The sutures 18 are also made from biologically compatible materials, such as those mentioned for the sheet 12, and are preferably monofilament sutures.

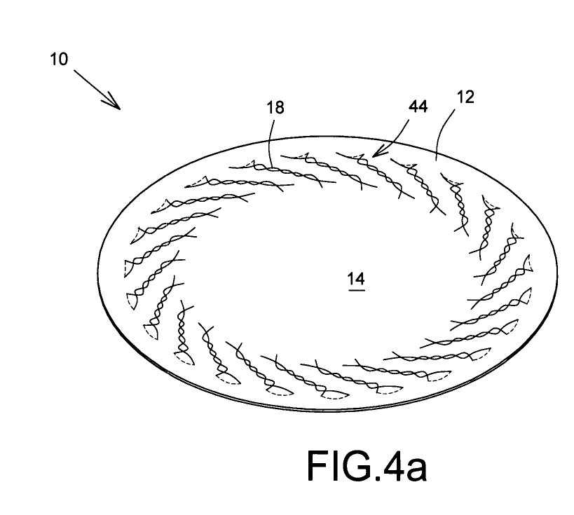

Having described the general characteristics of the patch 10, the deployment thereof for use in an underlay repair procedure for an aperture 20, such as a ventral hernia, is now described with reference to FIGS. 2 and 3. Initially, the patch 10 is positioned with the sheet 12, and preferably the first sheet surface 14, proximal the first tissue surface 24 and extending under and toward the aperture 20. The sutures 18 (end 34a, 34b pairs as shown) are then extended, i.e. drawn, through the tissue 22, from the first tissue surface 24 therethrough and out of the second tissue surface 26. The drawing of the suture 18 through the tissue 22 may be effected, for example, by inserting a conventional suture passer (or through wire instrument)—not shown—through the tissue 22 from the second tissue surface 26 through the first tissue surface 24, engaging the suture 18 therewith, and drawing the suture 18 therewith through the tissue 22 from the first tissue surface 24 toward and out of the second tissue surface 26. Each suture end 34a, 34b is then pulled until the first sheet surface 14 locally and adjacently abuts the first tissue surface 24 while covering the aperture 20. Suture ends 34a, 34b (preferably of a same suture 18) are then attached to one another adjacent the second tissue surface 26 to locally fasten the sheet 12 to the tissue 22 with the sheet 12, and notably the first sheet surface 14, under covering the aperture 20.

Advantageously, since the sutures 18 are already connected to the sheet 12, there is no need, unlike with conventional patches, to use a needle or other surgical tool to thread the suture 18 from the first sheet surface 14 through the sheet 12, and possibly out through the second sheet surface 16, and then back through the sheet 12 out of the first sheet surface 14 to connect the suture to the sheet 12. Accordingly, the surgical procedure of repairing the aperture 20 with the patch 10 of the present invention is facilitated and the amount of time required to perform the procedure, compared to conventional patches, is reduced. Further, the risk of damaging other tissue or internal organs in proximity to the surrounding tissue 22 by inserting a needle or other instrument through the patch, as required with conventional patches, is eliminated. The elimination of this risk also reduces the risk of infection and of complications. In addition, as the sutures 18 are already attached to the patch 10 in a spaced apart relationship around the perimeter 28 (at between about 0.5 cm (0.2 inch) and about 2.5 cm (1 inch), and preferably about 1 cm (0.4 inch) therefrom), the risk of irregular stitching, non-uniform placement or attachment of the sutures 18 to the patch 10 and tissue 22, which may be encountered with conventional patches, is reduced and proper placement of the patch 10 relative the tissue 22 and aperture 20 is facilitated.

Referring to FIGS. 1 and 3, for the embodiment shown, both suture ends 34a, 34b of a same suture 18 are spaced apart relative one another at a distance d1 varying between about 5 mm (0.2 inch) and about 10 mm (0.4 inch). Similarly, adjacent suture ends 34a, 34b from adjacent sutures 18 are spaced apart relative one another at a distance d2 varying between about 0 mm (0 inch) and about 10 mm (0.4 inch), and preferably at about 7-8 mm (0.3 inch). These distances d1, and especially d2, are intended to ensure the uniformity of the patch attachment and that each suture end 34a can be readily engaged with a suture passer and pulled through the tissue 22 for attachment to another, preferably adjacent, suture end 34b for securely connecting the sheet 12 to the tissue 22 with the sutures 18 relatively evenly distributed therearound. More specifically, and as shown in FIGS. 1 and 3, the sutures 18 typically form pairs, shown generally as 30, of adjacent suture ends 34a, 34b. Each pair 30 of adjacent suture ends 34a, 34b consists of a thread 32 of biologically compatible material, typically non-absorbable. Each thread 32 is threaded through the sheet 12 with an intermediate portion 36 of the thread 32 extending across a portion of the second sheet surface 16 and the first and second ends 34a, 34b extending out from the first sheet surface 14 and respectively forming the pair from a suture 18. However, one skilled in the art will appreciate that sutures 18 need not be connected to the sheet 12 in this fashion. In fact, each suture 18 could, if desired, be a single thread securely connected to, or having the intermediate portion 36 connected to the sheet 12 to one of the sheet surfaces 14, 16, or therebetween.

While the distances for the spacing of the sutures 18 described herein are well adapted for use of the patch 10 to repair apertures 20 such as ventral hernias, the spacing may be adapted, i.e. modified, in function of the size of the sheet 12 as well as the size of the aperture 20 to be repaired. For example, larger apertures may require larger sheets and greater, or less, spacing between sutures 18.

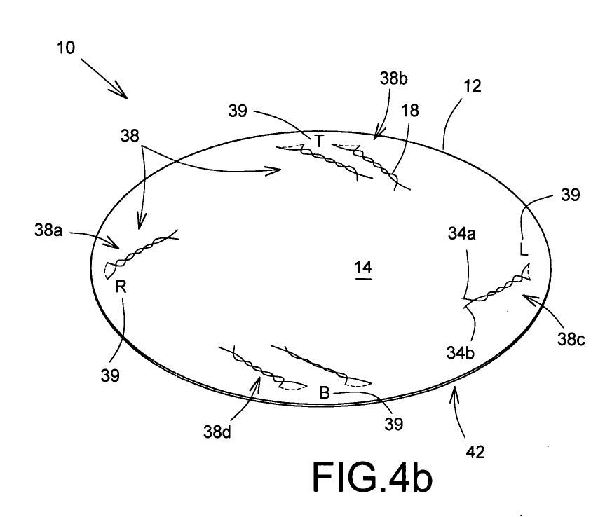

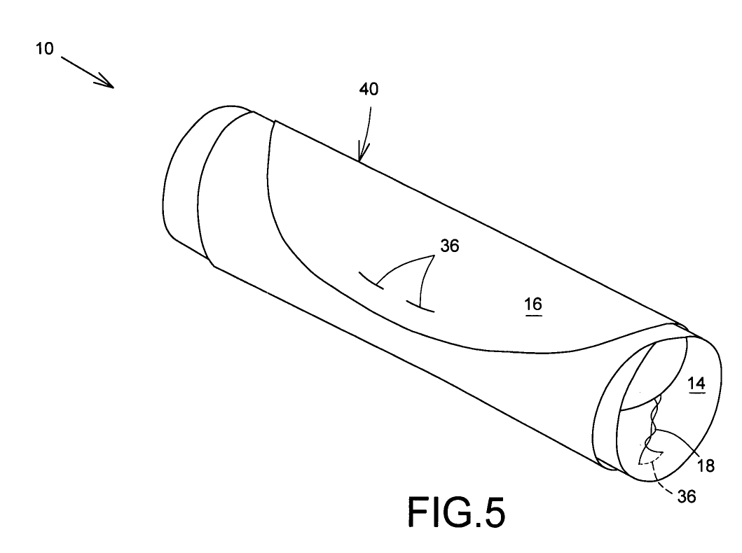

Further, sutures 18 could also be arranged in spaced apart groups 38, as shown in FIGS. 4b and 5, of at least one suture 18, each end 34 of each suture 18 of each group 38a, 38b, 38c, 38d being configured for attachment to the corresponding suture end 34b of a same suture 18 of the same group 38a, 38b, 38c, 38d. Each group 38a or 38b of suture 18 would, preferably, extend from the first sheet surface 14 at a position thereon substantially opposite an opposing group 38c or 38d, with the sheet 12 being connected to the tissue 22 via alternative means, such as, for example, stapling of or application of a biologically compatible adhesive to the sheet 12 at least in spaces extending between the groups. The use of multiple groups is especially useful the patch installation is made via laparoscopic treatment. To ensure proper orientation of the patch 10 relative to the aperture 20, the different groups 38 of sutures 18, typically opposite groups 38a, 38c and 38b, 38d on symmetrical patches, are visually identified using visual identifiers 39 such as different suture colors, suitable printed markings on the patch adjacent the groups (as dots, bars, letters T, B, L and R for top, bottom, left and right or N, S, E and W for north, south, east and west) and the like, as shown in FIG. 4b.

Reference is now made to FIGS. 4b and 5. Typically, the patch 10 is manufactured, packaged, or otherwise initially configured in a preferably compactly rolled first sheet configuration, shown generally as 40 in FIG. 5, in which the sheet 12 is compactly rolled, and sterilized and packaged in that first sheet configuration. The compact first sheet configuration 40 facilitates insertion of the sheet 12, obtained and/or taken out from the manufacturing package (not shown—a sterilized package may contain a plurality of patches 10 with preconnected sutures 18), into the body of the patient and placement of the sheet 12 in proximity to the aperture 20 and tissue 22. The sheet 12 may then be unrolled into the second sheet configuration, shown generally as 42 in FIG. 4b, for connection to the tissue 22 to under cover the aperture 20. The compact first configuration 40 is particularly useful for reducing the size of incisions required for inserting the patch 10 into the body of the patient, especially when the surgical procedure for repairing the aperture 20 with the patch 10 is performed laparoscopically.

Referring now to FIG. 2, typically, the sutures 18 are initially placed in a first suture configuration, shown generally as 44 in FIGS. 2, 4a and 4b, and in which the suture ends 34a, 34b are laid securely (typically releasably bonded), ideally partially folded, and rolled or twisted in corresponding pairs 30 (for improved identification thereof since the suture ends 34a, 34b could easily be about 15 to 20 cm (6-8 inches) long) across the first sheet surface 14, and preferably at least partially releasably secured or bonded thereto using a biologically compatible adhesive or the like. The patch 10 is then typically sterilized and packaged into that configuration with the suture pairs 30 laid on the first sheet surface 14. The suture ends 34a, 34b may then be extended into a second configuration, shown as 46 in FIGS. 1 and 2, for connection to the tissue 12. The first suture configuration 44, which may be combined with the first sheet configuration 40, advantageously facilitates placement of the patch 10 with the sutures 18 readily engageable in a known configuration, i.e. first suture configuration 44, thus facilitating engagement thereof with a medical instrument such as a suture passer for extending the suture ends 34a, 34b into the extended second suture configuration 46 for connection to the tissue 22. Typically, as partially illustrated in FIG. 2, the health professional, for the installation of the patch 10 once in proper position relative to the aperture 20, untwist a first suture pair 30 and extend the to suture ends 34a, 34b through the tissue 22 before attachment to one another with the unused portion thereof being cut away and discarded; and typically each suture pair 30 being connected to the tissue one after another (again color coding or the like visual identifiers 39 help the installation process). As with the first sheet configuration 40, the first suture configuration 44 is particularly useful when the surgical procedure for repairing the aperture 20 with the patch 10 patch is performed laparoscopically.

Although the present patch 10, and method of use thereof, have been described with a certain degree of particularity, it is to be understood that the disclosure has been made by way of example only and that the present invention is not limited to the features of the embodiments described and illustrated herein, but includes all variations and modifications within the scope and spirit of the invention as hereinafter claimed.

I claim:

1. A method for covering an aperture in an internal biological tissue extending therearound in a body of a patient with a prosthetic repair patch comprising a sheet of biologically compatible material and sutures preconnected and integral thereto, and having at least a respective suture longitudinal end extending from a first sheet surface of said sheet and releasably securely laid thereacross, the method comprising the steps of:
a) obtaining the prosthetic repair patch having the sheet of biologically compatible material and sutures preconnected thereto and integral therewith;
b) positioning said sheet proximal a first tissue surface of the tissue in the body with said first sheet surface facing the first tissue surface and said sheet extending under the aperture, the first tissue surface generally facing away from a person installing said patch; and
c) securing said sheet to the tissue.

2. The method of claim 1, wherein the step of obtaining the patch includes taking the patch having the sheet and sutures preconnected thereto and integral therewith out from a sterilized manufacturing package.

3. The method of claim 2, wherein the sterilized patch is in a rolled configuration inside the package.

4. The method of claim 2, wherein the step of positioning said sheet includes unrolling the sheet from a compact rolled first sheet configuration into an unrolled second configuration with said first sheet surface facing the first tissue surface.

5. The method of claim 1, wherein said patch includes a visual identifier connected thereto, and wherein the step of positioning said sheet includes visually identifying said visual identifier to orient said sheet relative to the tissue and to the aperture thereunder.

6. The method of claim 1, wherein the step of securing the sheet to the tissue includes the steps of:
c) extending each said suture end through the tissue and out from a second tissue surface of the tissue generally opposite the first tissue surface;
d) pulling each said suture end until said first sheet surface locally and adjacently abuts the first tissue surface while under covering the aperture; and
e) attaching each said suture end with another said suture end adjacent the second tissue surface to locally fasten said sheet to the tissue.

7. The method of claim 6, wherein the step of extending each said suture end includes extending said suture end from a first suture position in which said suture end is securely laid across said first tissue surface into a second suture position in which said suture end is extended for connecting to the tissue.

8. The method of claim 7, wherein said suture ends are arranged in pairs and twisted to one another adjacent said first sheet surface when in said first suture position, the step of extending each said suture end further including, for each said suture pair, the step of:
untwisting said suture pair while extending corresponding said suture ends from said first suture position into said second suture position.

9. The method of claim 7, wherein said suture ends are arranged in pairs and rolled adjacent said first sheet surface when in said first suture position, the step of extending each said suture end further including, for each said suture pair, the step of:
unrolling said suture pair while extending corresponding said suture ends from said first suture position into said second suture position.

10. The method of claim 6, wherein the step of extending each said suture end comprises, for each said suture end, the steps of:
c1) inserting a suture passer through the tissue from the second tissue surface through the first tissue surface for engaging said suture end therewith; and
c2) drawing the suture end through the tissue with the suture passer from the first tissue surface toward and out of the second tissue surface.

11. The method of claim 1, wherein the sutures are at least partially folded, and rolled or twisted in corresponding pairs across the first sheet surface.

12. The method of claim 11, wherein the sutures are at least partially releasably bonded onto and across the first sheet surface.

13. The method of claim 1, wherein the patch having the sheet and sutures preconnected thereto and integral therewith are sterilized prior to packaging thereof.
